(19) United States
(12) Patent Application Publication
(10) Pub. No.: US 2024/0394801 A1
(43) Pub. Date: Nov. 28, 2024

(54) MACHINE-LEARNING ANALYSIS OF MEDICAL CLAIM PAY CLASS COVERATGE

(71) Applicant: GULFSTREAM OUTSOURCING AND SPECIALIZED BILLING, LLC, West Palm Beach, FL (US)

(72) Inventors: Richard FOSSIER, Milton, GA (US); Jatni BLANDON, Royal Palm Beach, FL (US); John DONAHUE, Plantation, FL (US); Douglas A. SHAMON, Palm Beach Gardens, FL (US); Joseph D. SHAMON, San Francisco, CA (US); Lorenzo Arturo SENCION, Miramar, FL (US)

(21) Appl. No.: 18/771,221

(22) Filed: Jul. 12, 2024

(51) Int. Cl. G06Q 40/08; G16H 15/00; G16H 50/70

(52) U.S. Cl. CPC G06Q 40/08; G16H 15/00; G16H 50/70

(57) ABSTRACT

The disclosed invention is directed to methods and systems for machine-learning analysis of medical-claim pay class coverage. The machine-learning analysis may predict whether a third-party payor class is the proper payor class for a medical claim sent to a health insurance company or directly to the patient. The system may communicate payor class determinations between devices in a computer network. A payor class determination may be based on a computed likeliness score from a trained machine-learning model. The analysis may include identifying medical codes from historical medical claims, standardizing the medical codes, screening the historical medical claims based on the medical codes, training a model based on the standardized medical codes and corresponding payor class determinations, and applying the model to new medical claims to generate predictions and determinations.

### TECHNICAL FIELD

The present disclosure relates in general to the field of machine-learning, and more specifically to the application of machine learning for analyzing pay class coverage for medical claims.

### BACKGROUND

Machine learning is an area of artificial intelligence that uses trained algorithms to produce adaptable models that can perform a variety of complex tasks. Machine learning relies on statistics to learn from data and generalize on unseen data. Often, when machine learning is used, the data the model is trained on, or the data the model is applied to, is so large that a team of humans could not practically (or sometimes, possibly) analyze the same data by hand. Sometimes, the machine-learning algorithms are so complicated that a human could not practically perform them by hand even on a small set of data.

Machine-learning algorithms are sometimes categorized based on being “supervised,” “unsupervised,” or “self-supervised.” Some algorithms include a combination of these learning categories, with large language models (LLMs) generally including both self-supervised and supervised learning. Generally, supervised machine learning involves training a model on inputs and outputs (labeled data); then, when applied to a new input, it predicts an output (a label). Generally, unsupervised machine learning involves training on unlabeled data, and involves creating clusters (groups) based on similarities between inputs. Generally, self-supervised learning involves training on unlabeled data, creating implicit labels for clusters, and providing predictions of labels (outputs) for new inputs.

A popular machine learning algorithm is called “logistic regression,” which is a supervised-learning algorithm that that is widely used for binary classification tasks and can provide outputs of probabilities on a scale between zero and one. Other types of supervised machine-learning algorithms for classification tasks include decision trees and k-nearest neighbors (KNN).

A large area of study in the field of machine learning is how to efficiently select and apply machine-learning algorithms to specific use-cases. Many use-cases benefit tremendously from these algorithms, though it is not obvious how to apply them to each to solve problems in those industries. Prediction accuracy through machine learning can far exceed that of humans, if implemented correctly, in addition to being faster and less expensive.

One industry that has had unsolved problems for years is insurance coverage that pays for medical claims. Medical expenses are sometimes covered by health insurance, sometimes covered by motor-vehicle insurance, sometimes covered by worker's compensation, sometimes covered by another type of policy, or sometimes covered by a combination of policies. Generally, both institutions (hospitals) and other medical providers send insurance companies medical claims for its patients, and request that the insurance companies pay those expenses. Insurance companies must analyze those medical claims and decide whether to pay the medical claim, to not pay the medical claim, or to request more information about the medical claim. Insurance companies need to efficiently analyze and address medical claims, and doing so with humans alone is slow, inefficient, and often inaccurate. There is a long-felt, unsolved need in the insurance industry to address claims for payment of healthcare incidents with machine learning, and identifying the correct way to do so may be highly profitable.

### SUMMARY

The disclosed invention is directed to methods and systems for machine-learning analysis of medical claim pay class coverage. The machine-learning analysis may predict whether a third-party payor or coverage type is the proper payor or coverage type for a medical claim sent to a health insurance company or directly to the patient. The systems may communicate payor class determinations between devices in a computer network. A payor class determination may be based on a computed likeliness score from a trained machine-learning model. The analysis may include identifying medical codes from historical medical claims, standardizing the medical codes, screening the historical medical claims based on the medical codes, training a model based on the standardized medical codes and corresponding payor class determinations, and applying the model to new medical claims to generate predictions and determinations.

The machine-learning analysis of medical claim coverage may comprise a method performed by one or more computer processors, the method comprising: receiving medical data associated with a plurality of historical medical claims; identifying, from the medical data, two or more medical codes for each of the historical medical claims; identifying, from the medical data, one or more payor classes for each of the historical medical claims; training a model based on the medical codes and the one or more payor classes for two or more of the historical medical claims, wherein the training involves one or more machine-learning algorithms; receiving, from a first device, a new medical claim; identifying, at a second device, for the new medical claim, two or more new medical codes, wherein at least one of the new medical codes is the same as at least one of the historical medical codes; applying the trained model to the two or more new medical codes to generate a prediction for the new medical claim; and reporting, to the first device, a payor class determination, wherein the payor class determination is based on the prediction.

In some embodiments, the first device is associated with a first health insurer; and the method further comprises: confirming the payor class determination for the new medical claim; re-training the model based on the confirmation and the two or more new medical codes; receiving, at the second device, a medical claim from a third device; identifying, at the second device, for the medical claim from the third device, two or more medical codes; applying the re-trained model to the two or more medical codes for the medical claim from the third device; reporting, to the third device, a new payor class determination for the medical claim from the third device, wherein: the new payor class determination is based on an output from the applying the re-trained model, and the third device is associated with a second health insurer.

In some embodiments, the method further comprises, after the identifying the two or more codes for each of the historical medical claims, and before the training the model: assessing, with a rules-based algorithm, for each of the historical medical claims, whether one or more of the medical codes for the historical medical claim indicate a certainty or a high likelihood of a third-party payor class for the medical claim; screening, from a selection of historical medical claims used for the training the model, the historical medical claims that contain the one or more medical codes that indicate the certainty or a high likelihood of a third-party payor class, wherein the training the model is based on the medical codes and the one or more payor classes for each of the historical medical claims that are not screened.

In some embodiments, the one or more payor classes for the historical medical claims is selected from options related to workers-compensation insurance, or motor-vehicle insurance; the generated prediction comprises a probability that the proper payor class for the medical claim is workers-compensation insurance, or motor-vehicle insurance; and the payor class determination is selected from options comprising an indication of whether a the proper payor class is workers-compensation insurance, or an indication of whether the proper payor class is motor-vehicle insurance.

In some embodiments, the method further comprises: prior to the applying the trained model, determining that the new medical claim is related to a prior medical claim; and grouping the two or more new medical codes with one or more medical codes from the prior medical claim, wherein the applying the trained model is performed on the two or more new medical codes in combination with the one or more medical codes from the prior medical claim.

In some embodiments, the determination that the new medical claim is related to a prior medical claim comprises applying one or more algorithms that account for at least one of a date range between the new medical claim and the prior medical claim, or a relationship between the medical codes for the new medical claim and the medical codes for the prior medical claim.

In some embodiments, the identifying the two or more medical codes for each of the historical medical claims comprises: identifying one or more strings of between three and seven characters, following ICD formatting, in each historical medical claim; recording the identified strings; for each recorded string having seven characters, removing the seventh character; for each recorded string having dot notation, removing the period; and for each recorded string having three characters, adding one or more buffer characters to the end of the recorded string.

In some embodiments, the method further comprises: receiving a second new medical claim, wherein the second new medical claim has one or more medical codes; determining, through a rules-based algorithm, that the one or more second new medical codes correspond to one or more medical codes that were previously identified as having a strong association with a particular payor class determination; associating, based on the determination of the rules-based algorithm, the second new medical claim with the particular payor class; determining, based on the determination of the strong association, to not apply the trained model to the one or more medical codes of the second new medical claim; and providing, to the first device, an indication of the particular payor class for the second new medical claim.

In some embodiments, the two or more historical medical codes comprise at least one procedure ICD code and at least one injury ICD code; and the two or more new medical codes comprise at least one procedure ICD code and at least one injury ICD code.

In some embodiments, the one or more machine-learning algorithms comprise at least one of logistic regression, a tree model, or k-nearest neighbors.

In some embodiments, the method further comprises: training a second model and a third model based on the medical codes and the one or more payor classes for each of the historical medical claims, wherein the training the second model and the training the third model involves one or more machine-learning algorithms; applying the second trained model and the third trained model to the one or more new medical codes to generate a second prediction and a third prediction for the new medical claim; and determining, based on the prediction, second prediction, and third prediction, a collective prediction, wherein the reported payor class determination is based on the collective prediction.

In some embodiments, the method further comprises: receiving feedback on the payor class determination for the new medical claim; and re-training the model based on the new medical codes and the received feedback.

In some embodiments, the method further comprises communicating, to a payor associated with the payor class determination, information related to the medical claim; receiving, from the payor associated with the payor class determination, a response related to coverage; and based on the received response, determining whether the medical claim should be paid.

In some embodiments, the denial of payment comprises an indication that the claim is likely covered by one or more of a motor-vehicle-accident policy or a worker's compensation policy; and the approval of payment comprises an indication that the claim is likely not covered by one or more of the motor-vehicle-accident policy or the worker's compensation policy.

In some embodiments, the identifying the two or more medical codes for each of the historical medical claims comprises: identifying one or more strings of between three and seven characters, following ICD formatting, in each historical medical claim; recording the identified strings; for each recorded string having seven characters, removing the seventh characters; determining a number of occurrences for each recorded string; and based on a determination that the number of occurrences for one or more recorded strings is below a threshold, removing the last character of those one or more recorded strings.

In some embodiments, the generated prediction includes a numerical score related to a likelihood of a third-party coverage type or payor class being a proper payor class for the new medical claim.

The machine-learning analysis of medical claim coverage may comprise a system comprising one or more computer processors, wherein the one or more computer processors performs a method, the method comprising: receiving medical data associated with a plurality of historical medical claims; identifying, from the medical data, two or more medical codes for each of the historical medical claims; identifying, from the medical data, one or more payor classes for each of the historical medical claims; training a model based on the medical codes and the payor classes for two or more of the historical medical claims, wherein the training involves one or more machine-learning algorithms; receiving a new medical claim; identifying, for the new medical claim, two or more new medical codes, wherein at least one of the new medical codes is the same as at least one of the historical medical codes; applying the trained model to the two or more new medical codes to generate a prediction for the new medical claim; and reporting a payor class determination, wherein the payor class determination is based on the prediction.

In some embodiments, the method performed by the system further comprises: prior to applying the trained model, determining that the new medical claim is related to a prior medical claim; and grouping the two or more new medical codes with one or more medical codes from the prior medical claim, wherein the applying the trained model is performed on the two or more new medical codes in combination with the one or more medical codes from the prior medical claim, and wherein the determination that the new medical claim is related to the prior medical claim comprises applying one or more algorithms that account for at least one of a date range between the new medical claim and the prior medical claim, or a relationship between one or more of the medical codes for the new medical claim and one or more of the medical codes for the prior medical claim.

In some embodiments, the method performed by the system further comprises, after the identifying the two or more codes for each of the historical medical claims, and before the training the model: assessing, with a rules-based algorithm, for each of the historical medical claims, whether one or more of the medical codes for the historical medical claim indicate a third-party coverage type or payor class for the medical claim; and screening, from a selection of historical medical claims used for the training the model, the historical medical claims that contain the one or more medical codes that indicate the third-party coverage type or payor class, wherein the training the model is based on the medical codes and the one or more payor classes for each of the historical medical claims that are not screened.

In some embodiments, the identifying the two or more medical codes for each of the historical medical claims comprises: identifying one or more strings of between three and seven characters, following ICD formatting, in each historical medical claim recording the identified strings; for each recorded string having seven characters, removing the seventh character; and for each recorded string having three characters, adding one or more buffer characters to the end of the recorded string.

### BRIEF DESCRIPTION OF THE DRAWINGS

For a more complete understanding of the present invention and its advantages, reference is now made to the following description and the accompanying drawings, in which:

FIG. 1 is a diagram that depicts an overview of one or more embodiments of the invention;

FIGS. 2A, 2B are diagrams that depict a machine-learning process for analyzing medical payors according to one or more embodiments of the invention; and

FIGS. 3-9 are flowcharts of steps performed by various embodiments of the invention.

### DETAILED DESCRIPTION

The following detailed description includes exemplary embodiments of the disclosed invention. The figures illustrate some of those embodiments. The term “may” in relation to a feature described in this specification indicates that the feature can be present in some embodiments, and can be omitted in other embodiments: “may” is used to describe an example that might be present in some embodiments, and “may” is non-exhaustive. If an optional feature includes multiple components, each component can be independently included in an embodiment unless the specification indicates that two or more of the components must be coupled together. The term “or” in a string of two or more options means “and/or” unless the context indicates otherwise: the term “or,” when joining a string of two or more options, indicates that a combination of one or more of the options can be selected for an embodiment.

Common medical code types include ICD (international classification of diseases, World Health Organization), CPT (current procedural terminology, American Medical Association), and HCPCS (healthcare common procedure coding system, Center for Medicare and Medicaid Services). “Medical codes,” in this specification, may comprise codes from any of those three medical code systems, or may comprise codes from other medical code systems. While the figures (and descriptions of the figures) may sometimes specifically identify ICD codes, it is to be understood that those figures are illustrative of non-exclusive embodiments of the invention, and ICD codes may be replaced with other kinds of medical codes, which may include CPT or HCPCS codes, in some embodiments.

A “payor class” is one or more entities that might cover costs associated with a medical claim. A payor class may be a health insurer, a motor-vehicle insurer, a worker's compensation insurer, or other insurer. A payor class may be referred to as a “payor type.” As referred to herein, the term “payor” may refer to a specific insurance entity (e.g., Geico, Farmer's, Travelers, Medicare, or Cigna).

A “proper payor” is the payor who should pay the medical claim, based on priority (primary payor or secondary payor). For medical claims that have as their cause a motor vehicle accident or a worker's compensation incident, a health insurer is generally a secondary insurer, while motor-vehicle or worker's compensation insurers, for example, are generally primary insurers.

Determination of attribution to non-health payors comes before determination of coverage by the non-health payors. For example, an injury (and related medical claims) resulting from a car accident may be attributable to a motor vehicle payor. But if the injured person and those involved in the car accident do not have motor vehicle insurance, then the related medical claims might not be covered by a motor vehicle payor. This is a distinction between being a proper payor for a medical claim and providing coverage for a medical claim. Health insurers may wish to evaluate whether a medical claim is likely attributable to a third-party payor, and then to evaluate whether any corresponding entities provide coverage for the medical claim. This may save time and expense compared to first attempting to discover specific coverage to pay for the claim when the claim itself has not yet been determined to be a claim that was caused by a motor vehicle accident or a worker's compensation incident. This is especially true where a medical claim does not specifically indicate a cause of injury.

After determining that a third-party coverage type or payor class is the proper payor for a medical claim, a health insurer may wish to evaluate whether a third-party payor will cover the medical claim. As an example, evaluating whether there is coverage for a medical claim may include requesting motor-vehicle-insurance information from a healthcare provider upon a determination that motor vehicle insurers are the proper payor for a medical claim; then, upon receiving motor-vehicle insurance information, communicating to a corresponding motor vehicle insurer a request for reimbursement.

In some situations, a medical claim may be attributable to a primary payor (whether a medical or third-party coverage type) but not covered by that payor. For example, the patient's benefits under a third-party payor's insurance plan may already be exhausted, or the particular cause of injury underlying the medical claim may be outside the scope of coverage for the patient's policy. This may occur, for example, if a cause of injury is a car accident, which may make the medical claim attributable to a motor-vehicle payor, but the patient's individual car insurance policy does not cover the insured's own medical expenses.

A “third-party payor” (or “third-party insurer”) is a payor other than a health insurer. “Third-party coverage” may include all types of motor-vehicle accident coverage: for example, personal-injury protection, MedPay, bodily injury protection, or uninsured motorist or underinsured motorist coverages. “Third-party coverage” may include worker's compensation coverage. “Third-party coverage” may include boater's insurance. A “third-party payor” may refer to any entity that provides such coverages, or is otherwise a non-healthcare-payor. Healthcare payors (also referred to as “health payors” or “health insurers”) may include Medicare, Medicaid, United Healthcare, Aetna, Cigna, Blue Cross Blue Shield or a myriad of other companies offering medical insurance coverage. A patient may sometimes take the place of a health payor if, for example, the patient does not have health insurance: in such a situation, the patient would need to pay the medical expenses directly—for example, in the case where a third-party payor (e.g., motor-vehicle insurer or worker's compensation insurer) is not identified for a claim whose cause was either a motor vehicle accident or a worker's compensation incident.

Medical claims do not always specifically indicate a cause of injury. If a medical claim indicates that the cause of an injury is a motor vehicle accident, a car-insurance policy covering such an accident should reimburse the hospital or health provider for that bill where such coverage is found to be available and applicable. If a medical claim indicates that the cause of an injury is a workplace accident, then reimbursement should begin with worker's compensation or other workplace injury coverage or insurance where such coverage is found to be available and applicable. A health insurance company is often a secondary insurer in claims caused by either a motor vehicle accident or a worker's compensation incident, while a car insurance company is often a primary insurer, so health insurance companies can often have car insurance companies pay for injuries resulting from car accidents. Many claims that do not indicate a cause of injury end up with a health insurance company; and, without knowing the cause of the injury, health insurance companies often just pay the hospital or medical provider's reimbursement request (the medical claim). However, even if a hospital or medical provider's claim for payment does not list a cause of injury, there are other indicators on a medical claim that can be used to determine a cause of injury, or otherwise indicate that the medical claim can be paid by an automobile or work comp policy, for example, rather than a health insurance company.

There are many thousands of possible medical codes that can be listed on a medical claim. Medical claims often include several medical codes. Through machine-learning algorithms, correlations or patterns between medical codes and causes of injuries or likelihood of third-party coverage may be determined. The algorithms may correlate medical codes individually or as combinations. Combinations may be based on inclusion in the same medical claim, or in prior, related medical claims. The complexity of correlations between combinations of codes and outcomes may increase exponentially with the number of codes included in each combination.

The disclosed invention may help identify medical claims that should not be paid by a health insurance company, and may adjudicate medical claims faster, at lower cost, and with higher accuracy in identifying the proper payor class. The disclosed invention may identify policies that tend to reimburse higher amounts than health care policies: for example, auto insurance and worker's compensation policies tend to have higher maximum coverage amounts than health insurer policies. This may benefit both the health payor and the health provider.

The disclosed invention may determine a likelihood of a third-party payor class being responsible for a medical claim, which may help with determining whether to explore coverage options through entities (e.g., companies) associated with that payor class. For example, the invention may determine that, based on medical codes in a medical claim, the medical claim has an 80% chance of being attributable to a motor-vehicle payor. From that determination (a high probability of a third-party coverage type being a proper payor class), the invention and its further processes may send requests to one or more motor-vehicle companies to see if those companies will cover the medical claim. If one or more of the motor-vehicle companies will cover the medical claim, the health insurer who received the medical claim may deny the claim (and not pay the claim), or may seek reimbursement for the claim (e.g., if the health insurer already paid the claim prior to determining that there is third-party coverage).

As another example, the invention may determine that, based on medical codes in a medical claim, the medical claim has a 20% chance of being attributable to a motor-vehicle payor. From that determination (a low probability of a third party being a proper payor), the invention may determine that the health insurer who received the medical claim should not expend resources seeking coverage from third-party entities, but should instead pay the medical claim itself (or should not seek reimbursement).

By determining whether to seek coverage from third parties, and determining which types of third parties to seek coverage from, health insurers can save time and resources. By more accurately predicting which medical claims are likely to be covered by a third party, health insurers may pay less medical claims (or may be reimbursed more frequently on those already paid).

As another example, the invention may be applied by a medical provider (e.g., a hospital) to identify proper payors. By identifying that a medical claim may be attributable to a non-health payor, providers may have a higher portion of medical expenses covered.

The invention disclosed in this specification may improve upon existing machine-learning algorithms, methods, or systems. Machine learning can involve data collection, data preprocessing, model selection, model training, model tuning and optimization, and deployment. The invention may improve upon data collection, for example, by identifying the data most important for collection and training. The invention may improve upon data preprocessing, for example, by identifying how best to standardize the collected data to yield improved predictions. The invention may improve upon model selection, for example, by identifying one or more model types that are best for machine-learning analysis of medical insurance coverage, and how to implement or combine those models. The invention may improve upon model training, for example, by identifying inputs or labels for the training set. The invention may improve upon model tuning and optimization, for example, by identifying types of feedback implemented for retraining the system. The invention may improve upon model deployment, for example, by identifying how to best connect devices in a network for sending communications related to medical claim payor class identification or subrogation.

As a more concrete example of the improvements the invention may have over existing machine-learning algorithms, methods, or systems, in the invention (1) data selection may comprise identifying the medical codes on the medical claim, which are a series of letters or numbers that are sometimes separated by a period, or which medical claims to use for training; (2) data preprocessing may comprise standardizing the data by removing characters or punctuation; (3) model selection may comprise selecting logistic regression to provide a prediction between zero and one of a third-party payor class being the proper payor class for a medical claim, or may comprise using a KNN model for classification of being attributable or not attributable to a third-party payor class, rather than using a clustering algorithm for that part of the analysis; (4) model training may comprise using ICD codes as inputs, and prior determinations of whether a third-party payor class is likely to be the proper payor class for a medical claim as labels for those inputs, as part of a supervised learning approach; (5) model tuning and optimization may comprise determining that a prediction or determination was incorrect, then retraining the model based on that medical claim with a correct label; and (6) deployment may comprise a connection through an application-program interface (API) between the machine-learning analysis system and a health insurance company, or a connection between the machine-learning analysis system and a hospital or third-party insurer so the system can automatically request more information from the hospital or third-party insurer related to a medical claim based on a determination that a logistic regression score is below or above a threshold (e.g., between 0.4 and 0.6).

There may be advantages to selecting medical codes for training. Medical codes identify injuries or medical procedures, which may have correlations to causes of injury (also known as “mechanisms of injury”), especially when viewed in combination with other medical codes for a patient at the same time or at a previous, recent time. And the causes of injury may indicate whether third-party insurance companies may cover the medical bill. By using medical codes as input data, the model can determine the strengths of correlations between medical codes (or medical code combinations) and whether the medical claim is attributable to motor vehicle insurance or worker's compensation policies, for example. Medical codes related to a broken arm, for example, could relate to a car crash, workplace injury, or someone falling down one's staircase at home; but more specific codes for the arm break may be more common in different types of accidents, and combining that medical code with a code for a type of neck injury may make a third-party-payor class prediction stronger, while a combination with a medical code for a sprained wrist may make the third-party-payor class prediction weaker. A combination with a procedure code relating to a type of wrist surgery, for example, may also affect a prediction. The medical code, being in formulaic and non-narrative format, may be better for providing accurate computer-based predictions, than other types of information.

There may be advantages to removing characters from medical codes in pre-processing. In the current version of ICD (ICD-10), there are over 70,000 ICD-PCS procedure codes, and over 69,000 ICD-10-CM diagnosis (injury) codes. Because of the specificity of the codes, some of the codes may only appear once or a few times in even a very large dataset, so determining correlations or predictions for that code may be challenging or impossible. With each character in the ICD code, the injury or procedure indicated becomes more specific. ICD codes are hierarchical. Accordingly, by removing a character at the end of the code—a type of “code roll-up”—the injury or procedure specified becomes less specific, and determining correlations and making predictions from that data point becomes easier for limited datasets. Though, if too many characters are removed, the specified injury or procedure may become too generalized to yield an accurate prediction. Whether a character for an ICD code should be removed in training or in applying a model may be based on principal component analysis or on an analysis of the total number of times that code appears in the dataset.

Analogously, there are thousands of HCPCS codes, and currently 11,163 CPT codes (as of 2024). Code roll-ups may be similarly advantageous to HCPCS, CPT, and other types of medical codes. HCPCS and CPT codes each comprise five characters. In some embodiments, especially where quantity of training data (medical claims) is light, it may be advantageous to group HCPCS or CPT codes together to generalize injury types. HCPCS or CPT codes may be grouped, for example, based on code ranges.

There may be advantages to selecting logistic regression or other classification algorithms for analyzing medical claims. Supervised learning may be advantageous based on improved accuracy over unsupervised or self-supervised learning if the restraints are well known and defined, or may require less computation power than those other methods. Classification algorithms have an advantage in that they are good at predicting whether new data does or does not have a particular label, and may be based on the model being trained through supervised learning. Classification algorithms are good at conveying if there is likely to be motor vehicle insurance coverage (or not), or if there is likely to be workers compensation coverage (or not). Logistic regression, KNNs, and tree models are all examples of classification algorithms.

Logistic regression can provide a value between zero and one, which can convey not only a binary decision but a probability that the decision is correct. This may be advantageous because it can convey the strength of a prediction. For example, if a score is close to zero, the prediction may be strong that motor vehicle insurance does not cover the claim (or that motor vehicle insurance companies are not likely the proper payor class); and if the score is close to 1, the prediction may be strong that motor vehicle insurance does cover the claim (or that motor vehicle insurance companies are likely to be the proper payor class). If a prediction is not strong, the analysis system may determine that more information is needed, or that it otherwise cannot make an accurate determination. The zero to one scale may also allow the engineer to specify a threshold, based on the score, in which a health insurance company should seek coverage from a third-party payor class. For example, a health insurance company may wish for medical claims to request coverage from a third-party payor if there is at least an 80% chance (a score of 0.8) that the claim is attributable to a third-party payor class, rather than the probability merely being 51%. A health-insurance company may wish to specify that the system should request more information from a medical provider if, for example, a score is between 0.4 and 0.8, rather than merely making payment for having a probability of below 80 percent for a third-party coverage type or payor class being the proper payor. Companies may select different threshold values based, for example, on their risk tolerance or on market pressures.

There may be advantages to connecting the analysis system to the medical-insurance system, such as through an API or Internet connection. In some embodiments, the analysis system may be incorporated into the medical-insurance system, but there may be advantages to separating the systems. For example, by separating the systems, the analysis system may be more easily trained on data from multiple health insurance companies. Furthermore, the API may provide a more direct routing of a hospital's claims to the analysis system when the hospital submits a claim to an insurance company.

There may also be advantages to connecting the machine-learning analysis system to a hospital's system so that the analysis system can automatically request more information from the hospital related to a medical claim; for example, based on a determination that a logistic regression score is below or above a threshold (e.g., between 0.4 and 0.6). By providing network connections between the hospital's system and the analysis system, the analysis system can request more information from the hospital's system without human intervention based on the results of an analysis.

The invention may also improve upon existing applications of software to analyze or process medical claims. For example, the invention may comprise rules-based algorithms (possibly in combination with machine-learning based algorithms: for example, LLMs for analyzing narrative-style text) for filtering out claims that are facially either covered or not covered by a third party coverage type or payor class prior to assessing claims based on trained models (for example, using logistic regression on a model trained with ICD data). And such rules-based algorithms may be more efficient, tailored, or accurate than existing algorithms.

The invention may also improve upon the accuracy of medical claim determinations. For example, the invention may calculate scores for medical claims through a neural network or other machine-learning architecture that has calculated correlations between a web of medical codes, or combinations of medical codes, with third-party coverage determinations. Such scores may be more accurate and informative than a human's analysis of a medical claim without such calculated understanding of correlations between data points. Furthermore, by accounting for correlations between historical medical claims, and doing so possibly based on thousands or millions of prior medical claims, and possibly based on multiple medical codes for each medical claim, the machine-learning system may provide an entirely different analysis than can be performed by a human: for example, not just based on scale or accuracy, but based on actually being able to assess correlations or trends of this type, to provide numerical scores for such information, or to convey such information in an automated fashion or through an API.

The invention may have the benefit of reducing costs for a health insurance company by determining that other entities may have responsibility for medical bills. Traditionally, when a patient receives services at a hospital or another medical facility from a provider of medical services, the patient provides insurance information, and the hospital or medical provider requests reimbursement for the medical expenses from the insurance company. Medical expenses may be covered by a person's health insurance, by the person's motor vehicle insurance (e.g., car insurance), by a worker's compensation policy, or by another type of policy. By having a motor-vehicle, worker's compensation, or other policy cover the medical expenses, rather than by paying the medical claim itself, medical insurance companies can save money, and with the saved money they can increase profits or reduce the price of medical insurance paid by patients/consumers. The invention may be utilized by a health insurance company, for example, to assess whether a medical claim from a hospital should be rejected based on a likely presence of third-party coverage, and therefore require that the hospital seek a different avenue for reimbursement. The invention may utilize novel machine-learning and other techniques for improving this process.

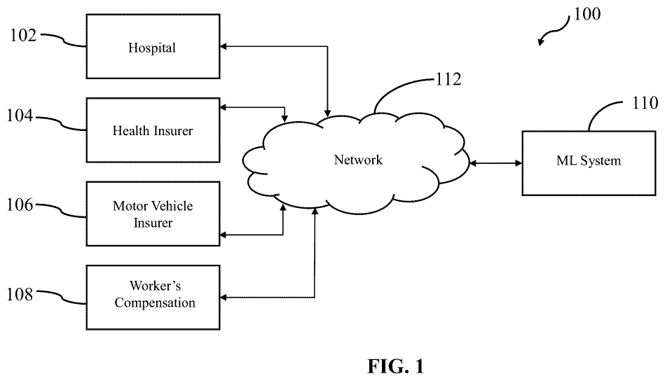

Turning to the figures, FIG. 1 is a diagram 100 that depicts an overview of one or more embodiments of the invention. The diagram 100 shows a hospital system 102 in communication with a health insurer system 104, a motor vehicle insurer system 106, a worker's compensation system 108, and an ML system 110 over a network 112. The diagram 100 also shows an ML system 110 on the right side, with each of the other systems on the left side. While each of the systems may communicate with other systems without going through the ML system 110, this depiction helps illustrate a focus on communications between the ML system 110 and the other four illustrated systems. The ML system 110 may perform the functionality described in this specification internally or based on its communications, directly or indirectly, with other systems connected to the network 112. Each of the systems connected to the network 112 may comprise one or more processors and a memory (such as SSD, HDD, or RAM). Each of the systems may connect to the network 112 through a wired or wireless connection. The network 112 may be the Internet, a LAN network, or a WAN network.

The hospital system 102 is a system associated with a hospital or other medical provider (e.g., a solo medical practice), such as a computer owned or operated by the hospital or other medical practice in which an employee at the hospital types and submits medical claims related to medical activities performed at or by the hospital or other medical practice or medical facility. It is to be understood that the term “hospital,” as used in this specification, may refer to other types of medical providers, or may be substituted with such other medical providers. The health insurer system 104 is a system associated with a health insurance company, such as a computer owned or operated by the health insurance company. A health insurance company may be referred to as a health insurer. The motor vehicle insurer system 106 is a system associated with a motor vehicle insurance company, such as a computer owned or operated by the motor vehicle insurance company that communicates motor vehicle insurance information for one or more covered persons to the network 112. The worker's compensation system 108 is a system associated with a worker's compensation policy, such as a computer that communicates worker's compensation information for one or more covered persons to the network 112.

The ML system 110 is a machine-learning system that can implement one or more of the methods described in this specification for analyzing medical-claim payors. The ML system 110 may, for example, receive data, identify features of the data, train a model based on those identified features, and apply the model to new data to make predictions about medical-claim payors. As a more specific example, the ML system 110 may implement logistic regression for a set of medical claims received from the health insurer system 104 to understand relationships between a large set of ICD codes and payor class determinations.

The ML system 110 may perform direct machine-learning functions, such as applying logistic regression; or may perform functions such as rules-based screening out of medical claims, which may not be part of a machine learning process. As examples, the ML system 110 may receive data, pre-process or standardize data, identify a training data set, train an ML model, further screen out claims, apply a trained ML model, identify feedback on outputs of the model, or retrain a model. Medical-code standardization may relate to the data pre-processing part of machine learning. Medical-claim screening may relate to the data-selection step of machine learning. Medical-claim screening may also be independent of machine learning (yet still performed by the ML system 110), for example, if it is based on a rules-based algorithm for excluding a new, unlabeled medical claim from being analyzed by an ML algorithm.

The ML system 110 is depicted as being separate from, and in communication with, the other systems. In some implementations, the ML system may be in communication with the health insurer system 104 via an application program interface (API) such that a hospital's interactions on a website maintained by a health insurance company at the health insurer system 104 may be directed to the ML system 110. In some embodiments, the ML system 110 may be incorporated into the health insurer system 104 or may otherwise be in the possession or control of a health insurance company. Connecting the ML system 110 to the health insurer system 104 may be advantageous in implementations where the ML system 110 helps a health insurance company associated with the health insurer 104 determine whether to accept or deny health insurance claims. Connecting the ML system 110 to the health insurer system 104 via API may be advantageous so that a hospital interacting with a website of the health insurance company can more easily interact with the ML system 110 than if the ML system 110 and health insurer system 104 were not so closely linked, but may also provide the advantage of being able to be linked to health insurer systems of more than one health insurance company. For example, in some implementations, there is an API link between the ML system 110 and the health insurer system 104, as well as an API link between the ML system 110 and a second health insurer system.

In some implementations, the ML system 110 may communicate with the hospital system 102 directly, or may be incorporated into the hospital system 102. While the health insurer may benefit from not paying claims that may be paid by another entity, the hospital also has an interest in ensuring its medical claims are paid, and some insurance policies may have more coverage than health-insurer policies. Accordingly, the health insurer and the hospital (or other medical provider) may benefit from the ML system and wish to implement it.

In some embodiments, secure file transfer protocol (SFTP) is used between the ML system 110 and the health insurer system 104. In some implementations, an SFTP may have some of the same advantages as API, or may promote a better user experience or processing time compared to APIs. SFTP may be implemented by a hospital or health insurer uploading files (medical claims) to a location in the network 112 that the ML system 110 can access; then, the ML system 110 may automatically detect that a file has been uploaded to the SFTP and upload it to a database associated with the ML system 110 in the form of a table containing line-item information from each medical claim.

The ML system 110 may evaluate medical claims to determine which payor classes it is likely attributable to. In some embodiments, the hospital system 102 may send a medical claim to the health insurer 104 through the network 112, that medical claim may be received by the ML system 110, which may assess whether the health insurance company associated with the health insurer system 104 should accept or deny the medical claim, or should request coverage information from the motor vehicle insurer system 106 or from the worker's compensation system 108. In some embodiments, the hospital system 102, or the health insurer 104, may request coverage information about a patient from the motor vehicle insurer system 106 or worker's compensation system 108, and determine whether policies associated with those systems for a patient will cover a medical claim.

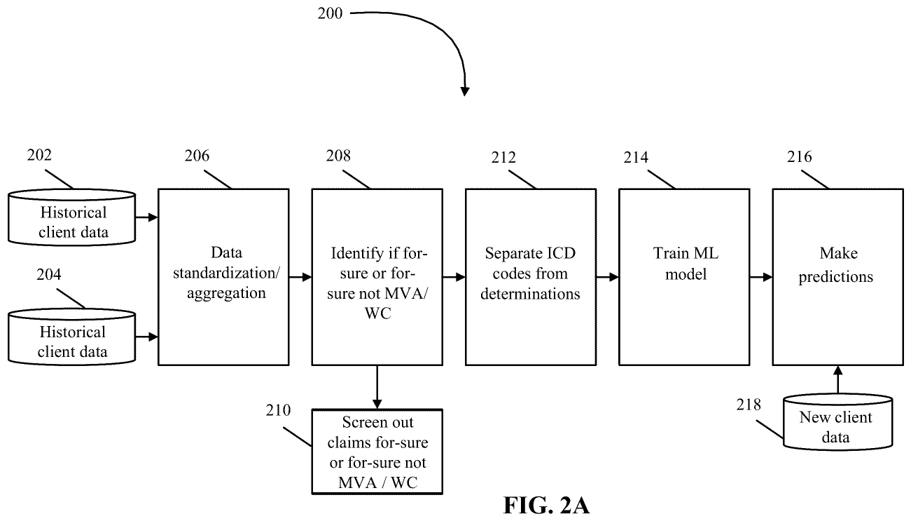

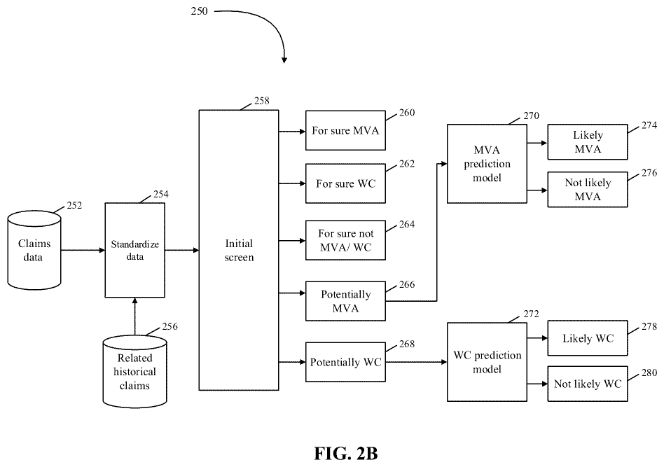

FIG. 2A is a diagram 200 that depicts a machine-learning process for analyzing medical claims according to one or more embodiments of the invention. Items 202, 204, 206, 208, 210, 212, and 214 relate to creating a trained model; and items 216 and 218 relate to applying the trained model to new data to generate a prediction. The diagram 200 depicts historical client data 202, 204 entering the system. Then, at 206, the system standardizes and aggregates that data. Then, at 208, the system identifies if claims are for-sure motor-vehicle accidents (MVA) or for-sure worker's compensation (WC) claims, based on various criteria. If the system detects claims in the historical data that are for-sure motor-vehicle or worker's compensation claims, those claims are removed from the data set at step 210. Then, at step 212, the system separates ICD codes from determination labels to facilitate training of the system. Then, at step 214, the system trains the model based on those ICD codes and determination labels. Then, at 218, new client data enters the system; and at 216, the system makes predictions of determination labels for that new client data. As noted earlier, in some embodiments, types of medical codes other than ICD codes may be used: for example, some embodiments may comprise HCPCS codes or CPT codes.

The historical client data 202 may comprise one or more medical claims obtained from a first database, such as a database of a hospital or medical insurance company. The historical client data 204 may comprise one or more medical claims obtained from a second database, such as a database of a hospital or medical insurance company. In some embodiments, historical medical claims data may be obtained from a research institution, such as the Massachusetts Institute of Technology (MIT). The data may enter the system through SFTP, API, or other type of data transfer method.

Standardizing the data at step 206 may comprise identifying ICD codes for each medical claim. Standardizing may comprise having each of the ICD codes be the same length by removing one or more characters from longer ICD codes, or adding place-holder characters—such as the letter X—for shorter ICD codes. Standardizing may comprise removing punctuation, such as periods, from ICD codes so that only letters or numbers remain.

Aggregating the data at step 206 may comprise grouping the ICD codes for each medical claim for medical claims containing two or more ICD codes. Aggregation may comprise storing all of the received medical-claim data into one table, such as a table comprising a column for ICD codes and a column for data labels. Data labels may comprise payor class determinations, such as a medical claim being attributable to a worker's compensation policy, attributable to a motor vehicle insurance policy, not attributable to worker's compensation, or not attributable to motor vehicle insurance. Data labels may comprise causation information, such as work-place injury, motor vehicle accident, not work-place injury, or not motor vehicle accident. The term “injury” is used broadly, and may cover a wide range of medical issues.

Identifying if for-sure MVA or for-sure WC at step 208 may comprise identifying information in a medical claim typed in by a medical professional at the hospital that indicates a cause of injury (e.g., stating that the injury occurred at work or in a car accident). Step 208 may comprise identifying, based on one or more ICD codes included in a medical claim, that a medical claim is for-sure MVA or for-sure WC if the correlation between the ICD codes and MVA or WC in historical data exceeds a threshold (e.g., the association between particular ICD codes and particular causations provides a greater than 95% confidence that new medical claims having those ICD codes is also from that type of causation).

For example, a machine-learning algorithm may recognize patterns in historical medical-claim data and determine that almost all medical claims having a combination of three particular ICD codes is the result of a motor-vehicle accident; then, at step 208, the presence of those three particular ICD codes may be identified for another medical claim; then, at step 210, that other medical claim may be excluded from the training data for the machine-learning model. As another example, if a medical claim has an ICD code for a type of liver failure, the probability of that medical claim being covered by motor vehicle insurance or a worker's compensation policy may be very low such that the medical claim can be deemed for-sure not WC and for-sure not MVA, and the medical claim can be screened out from the training data for the machine-learning model.

Through steps 208 and 210, the ML system may send only medical claims having an uncertain relationship between medical codes and third-party coverage along to training the model at step 214. A similar screening of claims may be performed to the new (unlabeled) medical claims received by the system at step 218 to make a determination for the medical claim without applying the trained ML model to the medical claim.

Training the ML model, at step 214, may comprise logistic regression, for example, or one or more classification algorithms such as tree models or k-nearest neighbors (KNNs).

The new client data at 218 may comprise one or more new medical claims. Step 216 may comprise identifying ICD codes in the medical claims, standardizing and aggregating those ICD codes for each medical claim, or identifying if the ICD codes indicate a for-sure attribution to motor vehicle insurance or worker's compensation insurance for any of the medical claims. Receiving new client data at 218 may comprise receiving medical claims through SFTP or API. Making predictions at step 216 may comprise predicting a proper payor class for a new medical claim.

FIG. 2B is a diagram 250 that depicts a machine-learning process for analyzing medical claims according to one or more embodiments of the invention. The diagram 250 includes receiving one or more new medical claims, standardizing data from each medical claim, receiving related historical medical claims, providing an initial screening of claims, then applying a machine-learning model to make a prediction.

The diagram 250 begins at 252 with inputting medical claims data. The medical claims data 252 is new medical claims rather than historical medical claims, and the claims data 252 does not yet have labels or determinations as to cause, proper payor class, or coverage of an injury. Each medical claim in the claims data 252 may comprise one or more ICD codes. Medical claims data may comprise one or more medical claims from a hospital concerning one or more of the hospital's patients. The claims data may be provided to the system directly from the hospital system, from a health-insurer system, or from elsewhere within a computer network. The claims data may be received by the system via SFTP or API.

The system also receives data at 256 that comprises related historical claims data. The related claims 256 may comprise historical medical claims corresponding to one or more of the patients associated with one or more of the new medical claims. In some embodiments, the historical claims comprise third-party payor class determinations. Third-party payor class determinations include an indication of whether a third-party coverage type or payor class is a proper payor of a medical claim.

The system standardizes the data at step 254. Standardizing promotes comparison of data. Standardizing the data may comprise identifying one or more ICD codes in each claim, performing code roll-ups to reduce the number of characters in ICD codes, adding buffer characters to shorter ICD codes, or removing punctuation from the ICD codes. The standardizing of data may be performed on new claims or related historical claims.

The system continues to step 258 with an initial screening of the standardized data. The initial screening may reduce the number of claims that need to be analyzed by a machine-learning model. The initial screening may be based on the standardized data for the new claims alone or in combination with the standardized data for the related historical claims. As depicted, the initial screening results in five possible determinations for each new medical claim: for-sure motor-vehicle accident 260, for-sure worker's compensation 262, for-sure not motor-vehicle accident or worker's compensation 264, potentially motor vehicle accident 266, or potentially worker's compensation 268. The initial screen may be performed with a rules-based algorithm, such as based on keywords in a narrative, free-response styled portion of medical claim (e.g., “car crash”) or specifically-identified ICD codes. The initial screen may be performed based on a large language model (LLM) analyzing narrative, free-response-style language in a medical claim. The initial screen may be performed based on one or more ICD codes in a medical claim, which may be specifically identified by a programmer of the system, or may be identified through a pattern-recognition determination by the system after analyzing many ICD codes and coverage determinations or coverage predictions. The initial screening may be based on other types of medical codes.

An indication that a medical claim is “for sure” caused by a motor vehicle accident, or “for sure” covered by motor vehicle insurance may indicate a high level of certainty, such as 95% or 99%. For example, liver damage or cancers may be deemed “for sure not MVA/WC” at 264, based on those injuries generally not being caused by motor vehicle accidents or work injuries, but in some rare circumstances (such as carcinogen exposure during an accident or while at work), this filtering may result in an incorrect determination; to account for such circumstances, the system may provide a hospital or medical provider an option for providing more information related to the medical claim, or the system may allow for an appeal.

For HCPCS codes, for example, a claim might be categorized as for-sure not MVA and for-sure not WC at 264 if the code includes an “R” (indicating that the injury location is residential).

As another example, for some medical codes, the letter V in the code may indicate “vehicular” which would tend to be for-sure MVA; but V may be indicated for an injury resulting from a scooter accident, which might not be covered by an MVA policy. Accordingly, screening by the letter V in a code might not be appropriate. The letter V in a code may be more significant, though, based on correlations with other parts of the code (e.g., code 123456V may have a stronger correlation with injuries covered by MVA policies than code 123455V). In some embodiments, the letter may be removed (e.g., resulting in 123456 and 123455) in pre-processing of the data.

As depicted, only the medical claims with uncertain third-party coverage type or payor class attribution proceed to the MVA prediction model 270 or the WC prediction model 272. The other claims may be reported to a health insurance company or hospital, for example, such as by an indication that the claim is attributable or not attributable to motor-vehicle or worker's compensation policies (a payor class determination). For example, at 264, the ML system may return a determination to the health insurer that the claim should be paid because it is not attributable to a third-party policy.

At 260, the ML system may request coverage information from the health provider to identify applicable motor vehicle insurance policies for the medical claim (e.g., the patient's motor-vehicle insurer). Step 260 may also involve the ML system notifying the motor-vehicle insurer that it is to pay for the medical claim. Step 260 may involve—in addition to determining that motor-vehicle insurers are the correct payor for a medical claim—determining which specific motor-vehicle insurance company is the correct payor for the medical claim.

In some embodiments, the health insurer may pay the medical claim first, then request the ML system 110 to analyze the medical claim. In such embodiments, the determination sent from the ML system 110 to the health insurer may relate to whether the health insurer should seek reimbursement: for example, from the health provider, or from a third-party insurer (e.g., subrogation of the proper payor).

There may be advantages to screening medical claims. Medical claims may be screened from the training data prior to training a machine-learning algorithm; or may be screened from new, non-labeled data that is received by the machine-learning (ML) system. Medical claims may be screened based on rules-based algorithms that indicate, for example, that a medical claim is not appropriate for a machine-learning model. For example, some medical codes (or combinations of medical codes) have strong associations with payor class determinations (such as that there likely is a type of third-party coverage type or payor class attributable to the medical claim) such that an ML algorithm is not needed for assessing the claim. Such codes (or combinations of codes) may have a weight for the model that is very close to 1 or very close to 0 such that their inclusion in the model would make the model bulkier but not significantly more precise. Medical claims containing those codes or combinations of codes may be screened out of the training data. Non-labeled medical claims containing those codes or combination of codes may be screened out from being analyzed by the ML model. Whether a code or combination of codes should be screened out may be based on human involvement or based on correlations derived by an ML algorithm. Screening out medical claims may improve the functioning of the model, or reduce the number of medical claims that need to be sent to the ML model.

The two prediction models 270, 272 are separated based on motor vehicle accidents or worker's compensation. In some embodiments, one model may account for both MVA and WC. In some embodiments, there may be more than two models. As depicted, the output of these models 270, 272 is a prediction that a medical claim is likely MVA 274, not likely MVA 276, likely WC 278, or not likely WC 280. From these predictions, the system may determine that a health-insurance company will or will not pay for the medical claim (i.e., the claim is accepted or denied by the health insurer), that a health-insurance company should seek coverage information from a hospital or third-party insurer, or that a health insurance company should subrogate the medical claim to a third-party insurer. Likely MVA 274 may be a prediction indicating that the cause of the injury is likely from a motor vehicle accident or may be a prediction indicating that the medical claim is likely covered by a motor vehicle accident policy (e.g., a car insurance policy).

The system may help the health insurer deny claims that are covered by a third party (likely WC or likely MVA), but may recommend that the health insurer accept (pay) claims that are not likely covered by a third party (not likely WC and not likely MVA). The system may use thresholds for this determination, such as a score of 0.7 from a logistic regression model. The system may report these predictions to the hospital that sent the medical claim, or to the health insurance company that must determine whether to accept or deny the claim. The system may use the predictions to generate health-insurer payment determinations, such as “pay,” “do not pay,” or “request more information.” The system may request more information, or suggest a request of more information, based on scores being within a range (e.g., 0.3 to 0.7), based on a score being below a value (e.g., below 0.7), or based on scores being above a value (e.g., 0.3).

A prediction from the ML system may indicate that a third party coverage type or payor class is the proper payor class for a medical claim. The prediction may take the form of a numerical score or a categorical indication (e.g., “likely WC”). The ML system may send that prediction, as a payor class determination, directly to the health insurer, health provider, or third-party system. The ML system may also modify the prediction, such as by using a prediction of “0.8 MVA” to send a payor class determination of “likely MVA” to a health insurer, health provider, or third-party system. Furthermore, the ML system may modify a prediction of “likely MVA” to a payor class determination that indicates “request coverage information from a motor-vehicle company associated with the medical claim” or “request coverage information from the health provider to determine the motor vehicle company associated with the medical claim.”

A prediction is an output of a ML model. A payor class determination is what is sent from the ML system to other systems in the network (e.g., the health insurer's system, the health provider's system, or a system of a third-party payor) to be acted on by or at those other systems. A payor class determination is based on a prediction. A payor class determination may be the same as the prediction, or may be a modified version of the prediction.

An output of a prediction model may be based on more than one medical code in a new medical claim. If the prediction is based on more than one medical code, the system may provide information about how heavily each medical code affected the final decision. It may do so by analyzing each code individually, or by performing other analyses. The system may provide, as a prediction, for each medical claim, a corresponding score and a relative weight for each medical code. Providing weights for each code may improve re-training of the model, or may help with screening of subsequent medical claims (e.g., at step 258).

Payor class determinations may be used to re-train the model or to assist in initial screening 258. For example, a determination of “likely MVA” may be stored in a database in association with the ICD codes for the corresponding medical claim. If one or more of the ICD codes in that claim, each time they appear, after a number of appearances, result in that same determination, the system may provide in the initial screening that those one or more ICD codes when present in a medical claim should result in “for sure MVA” 260.

The system may also implement self-supervised learning (machine feedback), or may incorporate human feedback for purposes of retraining the model based on supervised learning on the new claim data. Human feedback may involve a human confirming or denying a prediction (e.g., that the claim is likely covered by MVA insurance, or that MVA is likely a proper payor) or a determination (e.g., that MVA is likely a proper payor, or that the health insurer should seek subrogation from a motor-vehicle insurer).

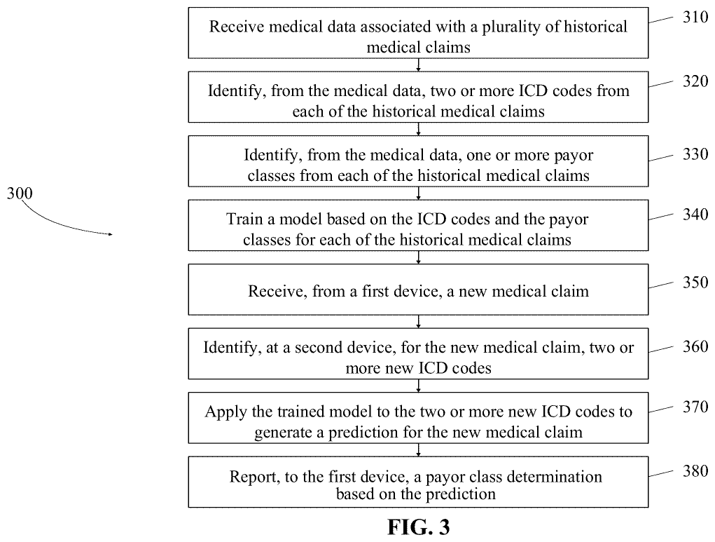

FIG. 3 is a flowchart 300 of steps performed in various embodiments of the invention. The flowchart 300 involves receiving historical medical claims, identifying ICD codes in the medical claims, identifying determination labels for the medical claims, training a model based on those identified characteristics, receiving a new medical claim, identifying ICD codes for that new medical claim, applying the trained model to those identified ICD codes, and reporting a payor class determination based on a machine-generated prediction. Though the flowchart specifically identifies ICD codes, in some embodiments, HCPCS or CPT codes, or other types of medical codes, may be implemented.

The flowchart 300 begins at step 310 with receiving medical data associated with a plurality of historical medical claims. The medical data may be obtained via SFTP, API, or other means for electronically receiving data. The historical medical claims may be received individually (e.g., transmitted in real time as they are verified) or as a set of several, hundreds, thousands, or even millions of medical claims. Historical medical claims may be entered electronically by an employee of a hospital and transmitted to a health insurer. Historical medical claims may be medical claims that have already been associated with a payor class determination. The historical medical claims each comprise at least one ICD code and at least one associated payor or payor class. Historical medical claims may be aggregated electronically by a company such as a health insurance company or a research institution (e.g., MIT). For example, the historical medical data may be organized in a data table, such as in an Excel spreadsheet or csv file.

The flowchart 300 continues to step 320 with identifying, from the medical data, two or more ICD codes from each of the historical medical claims. This step is related to compiling a data set to train a machine-learning model on. This step specifies at least two ICD codes instead of just one ICD code because correlations between single ICD codes and outcomes or determinations (e.g., that the medical claim is attributable to a third-party coverage type or payor class) may be screened out prior to applying an ML model (e.g., through a rules-based algorithm that screens for ICD codes having a strong association with a determination, as determined by an ML model) in some embodiments. Correlations between one ICD code and one or more payor classes are less complicated than the web of correlations that may be present between multiple ICD codes and the one or more payor classes. Accordingly, for this flowchart, only the historical medical claims having two or more ICD codes are included in the definition of historical medical claims; any medical claims received at step 310 having only one medical code are not included in the identified received historical medical claims.

The more ICD codes clustered for analysis (e.g., based on inclusion in the same medical claim), generally the more complicated the correlations. Some combinations may be frequent, and have predictable results (e.g., whenever a combination of two codes is included in a medical claim, the medical claim is attributable to a third-party payor). Sometimes one medical code is indicative of a determination, anytime included in a medical claim, regardless of whether there are other medical codes in the medical claim. An ML model may recognize such patterns, and use those patterns to screen out medical claims that include such individual or combinations of medical codes prior to training a model (i.e., so that the model is not trained on those medical claims) or prior to applying the model to new medical claims (i.e., so that the model is not used for those medical claims). An ML determination of for-sure or for-sure-not attributable to a third-party payor class, for use in screening, may be based on a threshold probability, such as 99 percent.

Screening out of medical claims that have one ICD code, or that are identified as otherwise being for-sure MVA, for-sure WC, for-sure not MVA or WC, or some other screening metric, may be performed at or around step 320 of the flowchart 300. Similarly, screening of new medical claims may be performed at or around step 360 of the flowchart 300.

In some embodiments, medical claims are not screened, and medical claims having only one medical code may be provided to the ML algorithm. But the benefits from the algorithm may be more apparent when two or more medical codes are involved, and the analysis becomes more complicated.

Identifying an ICD code may comprise identifying an injury code, mechanism of injury code, or a procedure code. Identifying ICD codes may comprise analyzing the medical document based on formatting, such as based on location (e.g., where ICD codes are generally located), style (e.g., what ICD codes generally look like), or context (e.g., in a box identified as being for procedure or injury codes). Identifying ICD codes may comprise identifying one or more columns in a table as being ICD codes.

In some embodiments, identifying a medical code includes identifying a medical specialty, such as whether a doctor is a dermatologist, cardiologist, OBGYN, etc. ICD codes are three to seven characters long, sometimes separated by punctuation such as a period (dot notation). The characters comprise letters or numbers. The more characters included for an ICD code, the more specific the injury or procedure is. More specific ICD codes may be more precise for predicting a cause of an injury or whether a third-party coverage type or payor class is a proper payor class for the injury. However, data sets based on very specific ICD codes might be too limited for generating strong correlations. Therefore, identifying ICD codes may comprise rolling up the codes such that seven-character codes are reduced to six characters, or such that six-character codes are reduced to five characters, etc., and identifying that five-or-six-character portion of the ICD code as the ICD code.

Identifying ICD codes may comprise standardizing steps such as removing punctuation from the ICD codes, such that the identified ICD codes do not have punctuation; or adding buffer characters, such as the letter x, to shorter ICD codes so that the identified ICD codes are all of the same length. In some instances, a medical claim may comprise twelve or more ICD codes.

The flowchart 300 continues to step 330 with identifying, from the medical data, one or more payor classes for each of the historical medical claims. The one or more payor classes for a medical claim are the one or more payor classes (e.g., categorized based on motor-vehicle, worker's compensation, healthcare) that are associated with a medical claim. The one or more payor classes may include a health insurer, a motor vehicle insurer, or a worker's compensation insurer.

Each payor may include multiple possible companies. For example, a motor vehicle insurer may include companies like Geico, Progressive, State Farm, Allstate, USAA, Liberty Mutual, Farmers, or Travelers.

The payor may be a “third-party payor” if it is an entity other than the entity that received the medical claim. A third-party payor may be an entity such as a car insurance company who a health insurer can pass a medical claim off to. If there is only one payor for a medical claim, and it is not a third-party payor, then the recipient of the medical claim (e.g., the health insurer) may be liable for the medical claim. If there is a third-party payor for a medical claim, then the recipient of the medical claim may be able to seek out whether a third-party payor company will cover the medical claim.

A payor class determination indicates one or more payor classes that are attributable to a medical claim. A payor class may be attributable to a medical claim if there is a strong association between the payor class and one or more aspects of the medical claim (e.g., medical codes in the medical claim). For example, there may be a strong association with a payor class if other medical claims having the same or similar medical codes are typically covered by a payor of that payor class. As another example, there may be a strong association with a payor class if the medical codes in the medical claim indicate an injury type, and that injury type is associated with insurance coverage often provided by payors of that payor class.

A payor class determination may indicate that there is or is not likely third-party coverage for the claim. A payor class determination may indicate that the medical claim is or is not likely to be covered under a motor vehicle accident insurance policy. A payor class determination may indicate that the medical claim is or is not likely to be covered under a worker's compensation policy. A payor class determination may indicate that a health insurance company should pay the new medical claim, or should request more information from a hospital, patient, or third-party insurer. In some embodiments, the payor class determination is confirmed by a human to facilitate supervised machine learning. In some embodiments, a payor class determination comprises two or more determinations.

The flowchart 300 continues to step 340 with training a model based on the ICD codes and the one or more payor classes for each of the historical medical claims. Training a model may comprise using logistic regression. Logistic regression may be helpful for classification of whether new medical claims do or do not have a likely third-party payor. Logistic regression may provide, for new medical claims, a probability on a scale from zero to one. Training a model may comprise using a classification algorithm such as tree models or k-nearest neighbors (KNNs). Training a model may comprise using more than one algorithm. In some embodiments, a system may train multiple models using different algorithms or different training data. Training the model may comprise separating data for the historical medical claims into a training set and a testing set. Training the model may comprise using payor class determinations as labels for ICD codes or groups of ICD codes. Training the model may comprise recognizing patterns or correlations in ICD codes independently or grouped together. For example, ICD codes may be analyzed together with other ICD codes included for the same medical claim.

The flowchart 300 continues to step 350 with receiving, from a first device, a new medical claim. A new medical claim is a medical claim that does not yet have a payor class determination. A medical claim may be a request for payment or reimbursement based on a patient's medical expenses, such as being based on one or more procedures or one or more injuries. A new medical claim may be filled in electronically and submitted by an employee of a hospital. The first device may be a device associated with a hospital who submits the new medical claim with a health insurance company for reimbursement. A new medical claim may be a standardized or filtered version of a typical medical claim, such as a list of one or more ICD codes that are associated with a medical claim. The first device may be associated with a health insurance company who sends the new medical claim to a machine-learning system for processing or analysis. The new medical claim may be received directly from the first device or indirectly from the first device.

The flowchart 300 continues to step 360 with identifying, at a second device, for the new medical claim, two or more new ICD codes. Identifying new ICD codes for the new medical claim may be similar to identifying the ICD codes for the historical medical claims. For example, identifying the ICD codes for the new medical claim may comprise identifying the standardized format of ICD codes (e.g., number of characters, no punctuation). The new medical claim may comprise ICD procedure codes or ICD injury codes. For example, the new medical claim may comprise two ICD procedure codes and three ICD injury codes. The two or more new ICD codes may be the same as one or more of the historical ICD codes. Optimally, the two or more new ICD codes appear many times in the historical data, alone or in combination, such that correlations or predictions are easier to determine for the new ICD codes.

The second device is a device that receives data for the new medical claim, and identifies ICD codes in that data. The second device may be a machine-learning (ML) system. The second device may receive the data formatted as a typical medical claim sheet provided to network by a hospital employee, or may receive the data formatted in table form, such as by having one or more rows for each new medical claim, and one or more columns for each associated ICD code.

The flowchart 300 continues to step 370 with applying the trained model to the two or more new ICD codes to generate a prediction for the new medical claim. Applying the trained model to the two or more new ICD codes may comprise applying the model to the new ICD codes in combination as a group. The prediction is an output of the model that relates to which payors are likely to be appropriate payors for the medical claim. A prediction may comprise a binary answer of yes or no, or may comprise a probability. For example, a prediction may be that yes, the medical claim is likely attributable to a third party payor; that yes, the medical claim is likely attributable to a motor-vehicle-accident insurance policy; that no, the medical claim is not likely attributable to a worker's compensation policy; or that there is a probability of 0.72 (72 percent) that the medical claim is attributable to a third party payor. Such a probability may be described as a score, and such a score may be used for creating a binary classification or other determination.

For new medical claims that have more than one ICD code, the ICD codes may be analyzed independently or as a combination. If analyzed independently, the model may be applied to each one to provide a prediction for each one. In such a situation, there may be more than one prediction, or the two or more predictions may be combined. For example, a system may implement an if-then step to determine that if any of the predictions is that there is likely a third-party payor, then that prediction is selected as the collective prediction. As another example, a system may select a highest probability or an average probability from a list of probabilities for a new medical claim. The system may also perform a vote between predictions to derive an ensemble or collective prediction. Combining predictions, or making determinations based on one or more predictions, may be performed in step 380.

The flowchart 300 continues to step 380 with reporting, to the first device, a payor class determination based on the prediction. The payor class determination may be reported through an electronic communication to the first device, such as through email, API, or SFTP. For example, a column in a table of a document stored in a network-accessible server may be updated to reflect the new determination. The payor class determination may be reported directly or indirectly to the first device.

A prediction is an output of a machine-learning model. A payor class determination is what is sent from the ML system to other systems in the network (e.g., the health insurer's system, the health provider's system, or a system of a third-party payor) to be acted on by or at those other systems, based on the prediction. The payor class determination for the new medical claim may be similar to the one or more payor classes for the historical medical claims. The payor class determination may be the prediction, or a modified expression of the prediction. The payor class determination may be that the claim is attributable to a third party payor, such as a worker's compensation policy or a car insurance policy. The payor class determination may be an indication to accept or deny the new medical claim, or that the health insurer is likely owed reimbursement or likely entitled to subrogate the medical claim.

The payor class determination may be based on applying a threshold to the prediction. As an example, if the prediction is a probability of 0.72 for the claim being covered by a car insurance policy, and a threshold was set at 0.70, the payor class determination may be to seek third-party payor coverage. As another example, under the same 0.70 threshold, if the prediction is a probability of 0.68, then the payor class determination may be to request more information. Requesting more information may result in an electronic communication being sent to a hospital associated with the new medical claim.

The payor class determination may be used to decide whether to, or how to, seek third-party payor coverage for a medical claim. After finding third-party payor coverage, a health insurer or ML system may make a determination of health-insurance payment. A determination of health-insurance payment relates to a health-insurer's payment (acceptance) or non-payment (denial) of a new medical claim, or of the health-insurer's entitlement to reimbursement or subrogation. A determination of health-insurance payment may indicate that the claim should be paid, that the claim should not be paid, or that the claim should have or should have not been paid. A determination of health-insurance payment may indicate that the medical claim is being accepted or denied based on third-party payor coverage of the claim. A determination of health insurance payment may indicate that a health insurer is entitled to reimbursement for a medical claim, or may be able to subrogate a different payor (e.g., that the health insurer is entitled to reimbursement from a car insurance company). In some embodiments, the payor class determination may be used as a label for the new ICD codes in a new training set for re-training the model.

A “particular payor” may be an identification of being a motor-vehicle payor, a worker's-compensation payor, or a medical insurer payor. An entity associated with a payor may be a company—such as Gieco, Allstate, or Traveler's Insurance—that is associated with a payor (e.g., a motor-vehicle payor type).

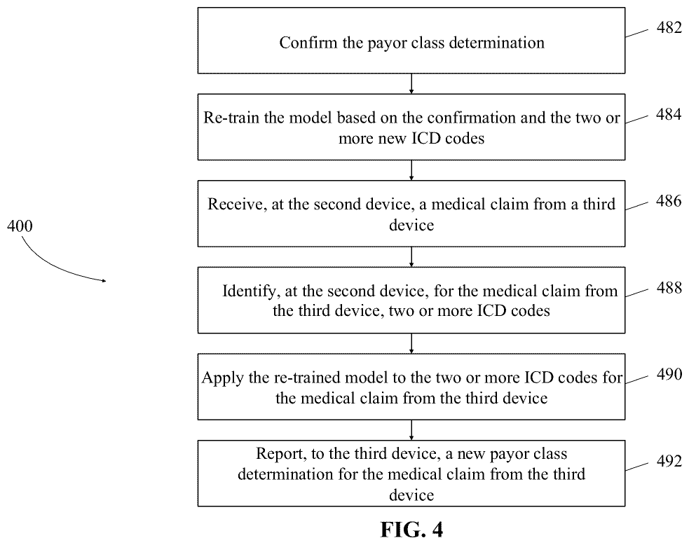

FIG. 4 is a flowchart 400 of steps performed in various embodiments of the invention. The flowchart 400 involves re-training a model based on a confirmed determination associated with a first device, then using that re-trained model for a prediction or determination associated with third device. This flowchart may illustrate that the ML system can interact with more than one health insurer, or that the ML system can interact with more than one health provider. This flowchart may illustrate that the ML system may be re-trained based on feedback.

The flowchart 400 begins at step 482 with confirming the payor class determination. Confirming the payor class determination may comprise comparing the payor class determination to a known result. For example, if the payor class determination is that the new medical claim is attributable to a motor-vehicle insurer, then the confirmation may be an indication that the payor class determination is correct or incorrect, that the medical claim is not attributable to a motor vehicle insurer, or that the claim is also attributable to a worker's compensation policy. Confirmation may be performed by a human, or may be performed through one or more other machine-learning models. Confirming the feedback may be based on a response from a health provider or an insurer based on having received the payor class determination.

The flowchart 400 continues to step 484 with re-training the model based on the confirmation and the two or more new ICD codes. Re-training the model may comprise adding a label to the two or more new ICD codes, similar to the payors class label used for the ICD codes of the historical medical claims. A label for the new ICD codes may be the payor class determination, if confirmed as being correct. Re-training the model may comprise adding the new ICD codes for the new medical claim into a training set for the model. Re-training the model may implement the same algorithm (e.g., logistic regression) or a different algorithm.

The flowchart 400 continues to step 486 with receiving, at the second device, a medical claim from a third device. This step may be similar to step 350 of flowchart 300. The third device may be another device on the network. For example, if the first device is a hospital's system, then the third device may be a second hospital's system.

The flowchart 400 continues to step 488 with identifying, at the second device, for the medical claim from the third device, two or more ICD codes. This step may be similar to step 360 of flowchart 300.

The flowchart 400 continues to step 490 with applying the re-trained model to the two or more ICD codes for the medical claim from the third device. This step may be similar to applying the trained model to the ID codes from the first device in step 370 of the flowchart 300.

The flowchart 400 continues to step 492 with reporting, to the third device, a payor class determination for the medical claim from the third device. This may be similar to the reporting the payor class determination to the first device of step 380 of the flowchart 300. The determination for the medical claim from the third device may be an output of the applying the re-trained model, or related to an output of the applying the re-trained model.

In some embodiments, instead of providing a determination, the system may provide the prediction directly. By providing the prediction rather than (or in addition to) a new determination for the third device, the second device may be informed of a score (i.e., likelihood, probability) for the medical claim from the third device, or may provide other information from which a determination may be derived.

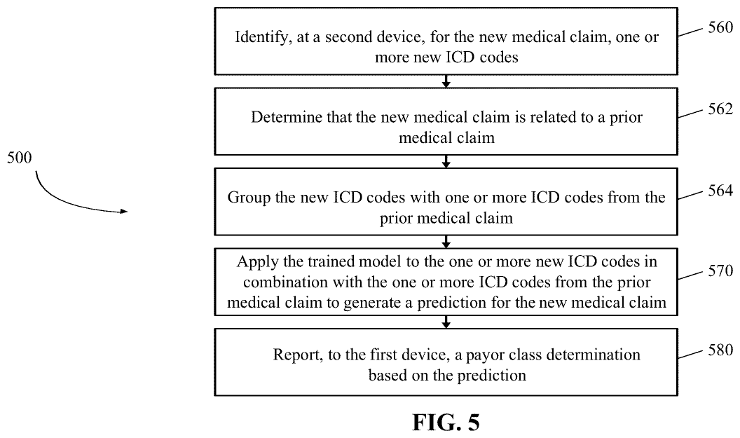

FIG. 5 is a flowchart 500 of steps performed in various embodiments of the invention. The flowchart 500 involves analyzing the medical claims based on ICD codes as combinations with other ICD codes appearing in related medical claims.

The flowchart 500 begins at step 560 with identifying, at a second device, for the new medical claim, one or more new ICD codes. This step may be similar to step 360 of flowchart 300.

The flowchart 500 continues to step 562 with determining that the new medical claim is related to a prior medical claim. Determining that a new medical claim is related to a prior medical claim may be based on determining that the patient for the new medical claim is also listed on a prior medical claim within a set time period (for example, 30 days). Determining that the new medical claim is related to a prior medical claim may be based on determining that the patient for the new medical claim is also listed on a prior medical claim, and that there is a relationship between the ICD codes for the new medical claim and the prior medical claim: for example, the new medical claim and prior medical claim may share one or more of the same ICD codes; or there may be a strong correlation between ICD codes (e.g., a procedure code in the new medical claim often follows from one of the injury codes listed in the prior medical claim). The system may implement one or more clustering algorithms to determine if a new medical claim is related to a prior medical claim, which may be part of something referred to as “claim attribution mapping.”

The flowchart 500 continues to step 564 with grouping the new ICD codes with one or more ICD codes from the prior medical claim. Grouping the ICD codes may be performed so that the ICD codes can be analyzed together in combination rather than just as independent items or events. Grouping the ICD codes together may be helpful for screening claims, or for applying a trained machine-learning model to claims.

The flowchart 500 continues to step 570 with applying the trained model to the one or more new ICD codes in combination with the one or more ICD codes from the prior medical claim to predict a coverage for the new medical claim. This step may be similar to step 370 of the flowchart 300, but further specifies that the ICD codes are analyzed in combination. The ICD codes being analyzed together may provide stronger correlations and more precise predictions than analyzing the ICD codes independently. Furthermore, by analyzing the ICD codes as a combination, there may be only one prediction for a new medical claim, so the system may not need to combine or select between predictions. Additionally, by analyzing the one or more ICD codes of the new medical claims with the one or more ICD codes of the prior medical claim, a score or prediction may be more precise or more likely to be accurate.

The flowchart 500 continues to step 580 with reporting, to the first device, a payor class determination based on the prediction. This step may be similar to step 380 of the flowchart 300.

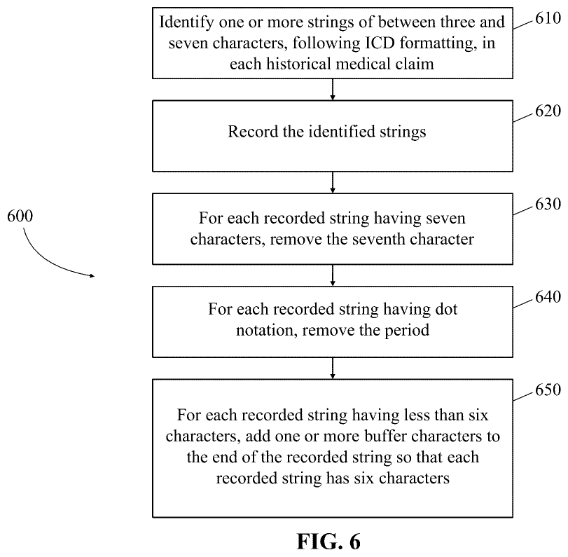

FIG. 6 is a flowchart 600 of steps performed in various embodiments of the invention. The flowchart 600 relates to identifying one or more ICD codes for a medical claim. The flowchart 600 involves identifying or standardizing ICD codes.

The flowchart 600 begins at step 610 with identifying one or more strings of between three and seven characters, following ICD formatting, in each historical medical claim. ICD codes are generally three to seven characters, with dot notation (a period between characters), with capitalized letters and no spaces between characters. The letters or numbers in ICD codes may appear random, and do not spell out dictionary words or phrases. In identifying ICD codes in a medical claim, a system may identify such strings or phrases by recognizing those characteristics of ICD codes, or may identify ICD codes in other ways, such as based on the location of the code in a document or a data table.

The flowchart 600 continues to step 620 with recording the identified strings. This step comprises saving, temporarily or long-term, the one or more ICD codes for a medical claim in the format that they were recognized in, or in a similar format. Saving these raw ICD codes may allow for the system to standardize the ICD codes for use in training a model. In some embodiments, identifying ICD codes may comprise listing standardized ICD codes.

The flowchart 600 continues to step 630 with, for each recorded string having seven characters, removing the seventh character. Removing the seventh character may help to generalize the most specific ICD codes, and to bring ICD codes closer to a uniform length. Removing the seventh character may not be necessary in some implementations, especially for data sets comprising many (e.g., thousands or millions) of medical claims having ICD codes, or for datasets having many (e.g., dozens, hundreds, thousands) of medical claims having a particular six-character string, where the additional specificity of ICD codes may promote precision and have enough data points to provide reliable predictions. Keeping too many characters may increase computation cost, and may not be necessary in implementations where the seventh character does not add significant precision or reliability.

The flowchart 600 continues to step 640 with, for each recorded string having dot notation, removing the period. Removing punctuation from ICD codes may help for processing the codes.

The flowchart 600 continues to step 650 with, for each recorded string having less than six characters, adding one or more buffer characters to the end of the recorded string so that each recorded string has six characters. Having each of the strings be the same length may help with processing. Because ICD codes become more specific with each character, an ICD code with four characters that match the first four characters of a five-character ICD code may be described as being a broader version of that four-character code. In some embodiments, a buffer character may be added to such a four-character code so that its corresponding medical claim can be more easily compared with a medical claim corresponding to the five-character ICD code. In some embodiments, a buffer character may indicate that the ICD code encompasses the more specific versions of the ICD code, which may aid in the comparison.

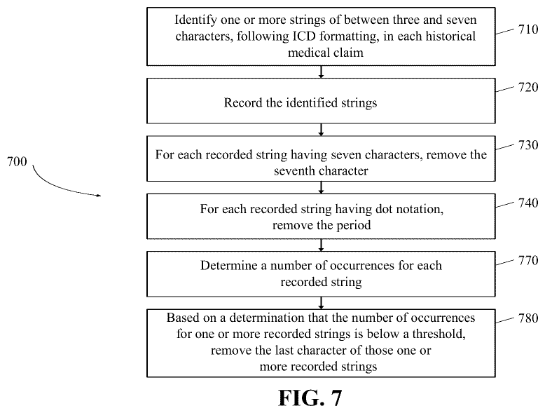

FIG. 7 is a flowchart 700 of steps performed in various embodiments of the invention. The flowchart 700 relates to identifying one or more ICD codes for a medical claim. The flowchart 700 involves identifying, standardizing, or rolling-up ICD codes.

The flowchart 700 begins at step 710 with identifying one or more strings of between three and seven characters, following ICD formatting, in each historical medical claim. This step may be similar to step 610 of the flowchart 600.

The flowchart 1000 continues to step 1020 with recording identified strings. This step may be similar to step 620 of the flowchart 600.

The flowchart 700 continues to step 730 with, for each recorded string having seven characters, removing the seventh character. This step may be similar to step 630 of the flowchart 600.

The flowchart 700 continues to step 740 with, for each recorded string having dot notation, removing the period. This step may be similar to step 640 of the flowchart 600.

The flowchart 700 continues to step 770 with determining a number of occurrences for each recorded string. For example, this step may determine that a string only appears once in the data set, that it appears several times, that it appears hundreds of times, or that it appears thousands of times.

The flowchart 700 continues to step 780 with, based on a determination that the number of occurrences for one or more strings is below a threshold, removing the last character of those one or more recorded strings. A threshold may be determined based on principal components analysis (PCA), based on a statistical analysis of the available strings (e.g., in the lower ten percent of strings), or based on a number set by a programmer as being a reliable predictor (e.g., at least five occurrences). The threshold may depend on the size of the historical data set. The threshold may be a whole number.

The feature of rolling up some of the strings may be useful because of the hierarchical nature of ICD codes, and how ICD codes refer to more specific conditions the more characters they have. More characters in an ICD code identifies more specific injuries, which may provide stronger correlations for predictions, and higher precision or accuracy. But when data sets are too small to have many instances of particular long ICD codes, reducing the specificity of those codes may provide a more reliable dataset by grouping the medical claims of similar higher-level codes. By keeping some ICD codes specific, where there are enough instances of the ICD code to be reliable, then generalizing some ICD codes where there are not enough instances of the ICD codes to be reliable, the model may optimize for reliability, precision, or accuracy.

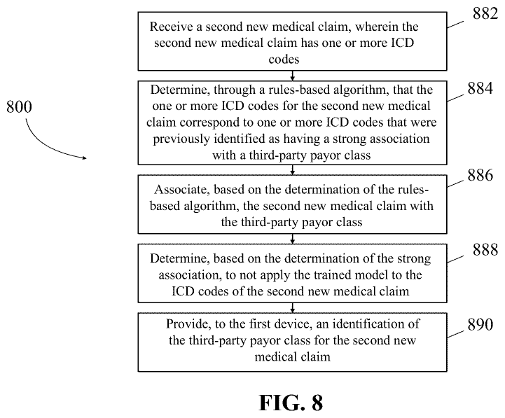

FIG. 8 is a flowchart 800 of steps performed in various embodiments of the invention. The flowchart 800 is directed to a process of screening out a medical claim without applying the trained machine-learning algorithm.

The flowchart 800 begins at step 882 with receiving a second new medical claim, wherein the second new medical claim has one or more ICD codes. This step may be similar to step 350 of the flowchart 300.

The flowchart 800 continues to step 884 with determining, through a rules-based algorithm, that the one or more ICD codes for the second new medical claim correspond to one or more ICD codes that were previously identified as having a strong association with a third-party payor. New ICD codes may correspond to prior ICD codes, for example, if the two ICD codes are the same, or if the new ICD code is a more specific version of the prior ICD code.

The previously identified ICD codes may be identified based on patterns recognized in a machine-learning algorithm (e.g., an injury code for a particular spine injury without the presence of an injury code for another type of spine injury, never resulted in a determination of third-party coverage after hundreds of medical claims listing that injury code, so the system flagged the code as categorically not covered by a third party when the other code is not present), or hard-coded by a human programmer (e.g., identifying a dermatology specialty, or dermatology-related injury or procedure codes, as always not covered by a third party, based on human knowledge of those types of injuries without needing pattern recognition).

If one or more medical codes indicate that a particular payor is a proper payor for a medical claim, and the particular payor is part of a particular payor class, then that indication may also include that the particular payor class is the proper payor class for the medical claim. Accordingly, there may be a strong association with the third-party payor class if there is a strong association with the third-party payor.

The flowchart 800 continues to step 886 with associating, based on the determination of the rules-based algorithm, the second new medical claim with the third-party payor. For example, if a new ICD code is the same as an ICD code identified as always attributable to a motor-vehicle payor, the new medical claim having the new ICD code may be associated with the determination of a motor vehicle payor being the proper payor class.

In some embodiments, the association may be between a medical claim and another type of determination, or with a prediction. As an example, if a new ICD code is the same as an ICD code identified as being for-sure not attributable to a third party payor, the system may associate the new ICD code with being for-sure not attributable to a third party payor.

The flowchart 800 continues to step 888 with determining, based on the determination of the strong association, to not apply the trained model to the ICD codes of the second new medical claim. This step may reduce the amount of computation needing to be performed by the system. If at step 886 it is determined that a code is for-sure not attributable to a third party payor, the system does not need to perform further analysis, and the system can report a result as a prediction, or as a determination, without needing to apply the trained model to the ICD codes for that medical claim.

The flowchart 800 continues to step 890 with providing, to the first device, the prediction for the second new medical claim. This step may be similar to step 492 of flowchart 400.

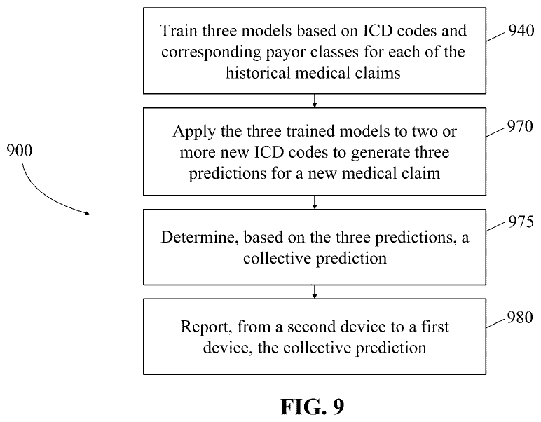

FIG. 9 is a flowchart 900 of steps performed in various embodiments of the invention. Flowchart 900 involves applying multiple trained models for a collective prediction.

The flowchart 900 begins at step 940 with training three models based on ICD codes and corresponding payor classes for each of the historical medical claims. This step may be similar to step 340 of the flowchart 300 except with three models. The models may be trained in series or in parallel. The models may be trained using the same machine-learning type (e.g., logistic regression), or with different machine learning types. For example, one model may use logistic regression, one model may use k-nearest neighbors, and one model may use a tree model. The models may use the same training data set or different training data sets.

The flowchart 900 continues to step 970 with applying the three trained models to two or more new ICD codes to generate three predictions for coverage for a new medical claim. This may be similar to step 370 of the flowchart 300 except performed three times (once for each model) and thereby providing three predictions. The predictions may each be a classification such as a yes or no for third-party payors, or may convey probabilities.

The flowchart 900 continues to step 975 with determining, based on the three predictions, a collective prediction. The collective prediction may be the result of a vote between the three models. For example, if two of the models vote yes, that the medical claim is likely attributable to a motor-vehicle-insurance payor, and the third model votes no, the “yes” vote may be the collective prediction. A collective prediction, for multiple predictions that provide scores, may be referred to as an ensemble score.

The flowchart 900 continues to step 980 with reporting, from a second device to a first device, the collective prediction. This may be similar to step 380 of the flowchart 300. In some implementations, the collective prediction itself is reported, and in some embodiments, a payor class determination based on the collective prediction may be reported.

1. A method performed by one or more computer processors, the method comprising:
receiving medical data associated with a plurality of historical medical claims;
identifying, from the medical data, two or more medical codes for each of the historical medical claims;
identifying, from the medical data, one or more payor classes for each of the historical medical claims;
training a model based on the medical codes and the one or more payor classes for two or more of the historical medical claims, wherein the training involves one or more machine-learning algorithms;
receiving, from a first device, a new medical claim;
identifying, at a second device, for the new medical claim, two or more new medical codes, wherein at least one of the new medical codes is the same as at least one of the historical medical codes;
applying the trained model to the two or more new medical codes to generate a prediction for the new medical claim; and
reporting, to the first device, a payor class determination, wherein the payor class determination is based on the prediction.

2. The method of claim 1, wherein:
the first device is associated with a first health insurer; and
the method further comprises:
confirming the payor class determination for the new medical claim;
re-training the model based on the confirmation and the two or more new medical codes;
receiving, at the second device, a medical claim from a third device;
identifying, at the second device, for the medical claim from the third device, two or more medical codes;
applying the re-trained model to the two or more medical codes for the medical claim from the third device; and
reporting, to the third device, a new payor class determination for the medical claim from the third device, wherein:
the new payor class determination is based on an output from the applying the re-trained model, and
the third device is associated with a second health insurer.

3. The method of claim 1, wherein the method further comprises, after the identifying the two or more codes for each of the historical medical claims, and before the training the model:
assessing, with a rules-based algorithm, for each of the historical medical claims, whether one or more of the medical codes for the historical medical claim indicate a third-party payor class for the medical claim; and
screening, from a selection of historical medical claims used for the training the model, the historical medical claims that contain the one or more medical codes that indicate the third-party payor class,
wherein the training the model is based on the medical codes and the one or more payor classes for each of the historical medical claims that are not screened.

4. The method of claim 1, wherein:
the one or more payor classes for the historical medical claims is selected from options related to workers-compensation insurance, or motor-vehicle insurance;
the generated prediction comprises a probability that a proper payor class for the medical claim is workers-compensation insurance, or a probability that the proper payor class for the medical claim is motor-vehicle insurance; and
the payor class determination is selected from options comprising an indication of whether the proper payor class is workers-compensation insurance, or an indication of whether the proper payor class is motor-vehicle insurance.

5. The method of claim 1, wherein the method further comprises:
prior to the applying the trained model, determining that the new medical claim is related to a prior medical claim; and
grouping the two or more new medical codes with one or more medical codes from the prior medical claim,
wherein the applying the trained model is performed on the two or more new medical codes in combination with the one or more medical codes from the prior medical claim.

6. The method of claim 5, wherein the determination that the new medical claim is related to a prior medical claim comprises applying one or more algorithms that account for at least one of a date range between the new medical claim and the prior medical claim, or a relationship between the medical codes for the new medical claim and the medical codes for the prior medical claim.

7. The method of claim 1, wherein the identifying the two or more medical codes for each of the historical medical claims comprises:
identifying one or more strings of between three and seven characters, following ICD formatting, in each historical medical claim;
recording the identified strings;
for each recorded string having seven characters, removing the seventh character;
for each recorded string having dot notation, removing the period; and
for each recorded string having three characters, adding one or more buffer characters to the end of the recorded string.

8. The method of claim 1, wherein the method further comprises:
receiving a second new medical claim, wherein the second new medical claim has one or more medical codes;
determining, through a rules-based algorithm, that the one or more second new medical codes correspond to one or more medical codes that were previously identified as having a strong association with a particular payor class;
associating, based on the determination of the rules-based algorithm, the second new medical claim with the particular payor class;
determining, based on the determination of the strong association, to not apply the trained model to the one or more medical codes of the second new medical claim; and
providing, to the first device, an indication of the particular payor class for the second new medical claim.

9. The method of claim 1, wherein:
the two or more historical medical codes comprise at least one procedure ICD code and at least one injury ICD code; and
the two or more new medical codes comprise at least one procedure ICD code and at least one injury ICD code.

10. The method of claim 1, wherein the one or more machine-learning algorithms comprise at least one of logistic regression, a tree model, or k-nearest neighbors.

11. The method of claim 1, wherein the method further comprises:
training a second model and a third model based on the medical codes and the one or more payor classes for each of the historical medical claims, wherein the training the second model and the training the third model involves one or more machine-learning algorithms;
applying the second trained model and the third trained model to the two or more new medical codes to generate a second prediction and a third prediction for the new medical claim; and
determining, based on the prediction, second prediction, and third prediction, a collective prediction, wherein the payor class determination is based on the collective prediction.

12. The method of claim 1, wherein the method further comprises:
receiving feedback on the payor class determination for the new medical claim; and
re-training the model based on the new medical codes and the received feedback.

13. The method of claim 1, wherein the method further comprises:
communicating, to an entity associated with the payor class determination, information related to the medical claim;
receiving, from the entity associated with the payor class determination, a response related to coverage; and
based on the received response, determining whether the medical claim should be paid.

14. The method of claim 1, wherein the identifying the two or more medical codes for each of the historical medical claims comprises:
identifying one or more strings of between three and seven characters, following ICD formatting, in each historical medical claim;
recording the identified strings;
for each recorded string having seven characters, removing the seventh characters;
determining a number of occurrences for each recorded string; and
based on a determination that the number of occurrences for one or more recorded strings is below a threshold, removing the last character of those one or more recorded strings.

15. The method of claim 1, wherein the generated prediction includes a numerical score related to a likelihood of a third-party payor class being a proper payor class for the new medical claim.

16. A system comprising one or more computer processors, wherein the one or more computer processors performs a method, the method comprising:
receiving medical data associated with a plurality of historical medical claims;
identifying, from the medical data, two or more medical codes for each of the historical medical claims;
identifying, from the medical data, one or more payor classes for each of the historical medical claims;
training a model based on the medical codes and the payor classes for two or more of the historical medical claims, wherein the training involves one or more machine-learning algorithms;
receiving a new medical claim;
identifying, for the new medical claim, two or more new medical codes, wherein at least one of the new medical codes is the same as at least one of the historical medical codes;
applying the trained model to the two or more new medical codes to generate a prediction for the new medical claim; and
reporting a payor class determination, wherein the payor class determination is based on the prediction.

17. The system of claim 16, wherein the method performed by the system further comprises:
prior to applying the trained model, determining that the new medical claim is related to a prior medical claim; and
grouping the two or more new medical codes with one or more medical codes from the prior medical claim,
wherein the applying the trained model is performed on the two or more new medical codes in combination with the one or more medical codes from the prior medical claim, and
wherein the determination that the new medical claim is related to the prior medical claim comprises applying one or more algorithms that account for at least one of a date range between the new medical claim and the prior medical claim, or a relationship between one or more of the medical codes for the new medical claim and one or more of the medical codes for the prior medical claim.

18. The system of claim 16, wherein the method performed by the system further comprises, after the identifying the two or more codes for each of the historical medical claims, and before the training the model:
assessing, with a rules-based algorithm, for each of the historical medical claims, whether one or more of the medical codes for the historical medical claim indicate a third-party payor class for the medical claim; and
screening, from a selection of historical medical claims used for the training the model, the historical medical claims that contain the one or more medical codes that indicate the third-party payor class,
wherein the training the model is based on the medical codes and the one or more payor classes for each of the historical medical claims that are not screened.

19. The system of claim 16, wherein the identifying the two or more medical codes for each of the historical medical claims comprises:
identifying one or more strings of between three and seven characters, following ICD formatting, in each historical medical claim;
recording the identified strings;
for each recorded string having seven characters, removing the seventh character; and
for each recorded string having three characters, adding one or more buffer characters to the end of the recorded string.

20. The system of claim 16, wherein the method performed by the system further comprises:
communicating, to an entity associated with the payor class determination, information related to the medical claim;
receiving, from the entity associated with the payor class determination, a response related to coverage; and
based on the received response, determining whether the medical claim should be paid.
